(19) United States
(12) Patent Application Publication
(10) Pub. No.: US 2024/0398433 A1
(43) Pub. Date: Dec. 5, 2024

(54) LITHOTRIPTER WITH DETECTION OF KIDNEY STONES

(71) Applicant: STORZ MEDICAL AG, Tägerwilen (CH)

(72) Inventors: Rafael Storz, Kreuzlingen (DE); Markus Belau, Konstanz (DE); Amin Mazloumian, Zürich (CH); Lukas Neuner, Effretikon (CH); Richard Weiss, Winterthur (CH); Matthias Rosenthal, Dielsdorf (CH)

(21) Appl. No.: 18/675,569

(22) Filed: May 28, 2024

(30) Foreign Application Priority Data
Jun. 5, 2023 (EP) 23177330.0

(51) Int. Cl. A61B 17/225

(52) U.S. Cl. CPC A61B 17/2255; A61B 17/2258

(57) ABSTRACT

A shock wave and/or ultrasound therapy system has an ultrasound and/or shockwave source with an ultrasonic transducer for ultrasonic imaging or an X-ray imaging system which is configured for providing a video signal, and which is coupled to a video processor. The video processor includes a neural network and is configured for detecting in the video signal at least one kidney stone and/or at least one kidney itself, which are marked in the video signal and displayed on a display. Position and/or orientation data of the at least one kidney stone and/or at least one kidney are delivered to a system controller for positioning the ultrasound and/or shockwave source

### PRIORITY CLAIM

This US Patent application claims priority to the pending European Application No. 23177330.0 filed on 5 Jun. 2023, the disclosure of which incorporated herein by reference.

### BACKGROUND

### 1. Field of the Invention

The invention relates to an extracorporeal shock wave or ultrasound therapy system, e.g., a lithotripsy system or Lithotripter for non-invasive treatment of stones, like e.g., kidney stones, urinary stones, gallstones, or other calculi within a mammal's body using acoustic pulses, or other applications of an extracorporeal shock wave or ultrasound therapy system.

### 2. Description of Related Art

Lithotripters, which generate high energy pulses to disintegrate concernments in a human body, may have shock wave and/or ultrasound sources within a reflector.

For localizing concernments to be treated, X-ray devices or ultrasound localizing systems using an ultrasonic transducer may be used. Such ultrasonic transducers may be handheld by a surgeon, or they may be fixed in a flexible or adjustable holding device. In EP 0 312 847 A1 a lithotripter with an ultrasonic transducer is disclosed, whereby the transducer is mounted at the center of the shock wave source. To allow for scanning a certain region around the focal point of the shock wave source, the transducer is made rotatable and tiltable within the shockwave source.

### SUMMARY

The embodiments of the invention provide a lithotripsy system that includes an ultrasound localizing system, which is configured to assist a user in detecting and localizing stones, e.g. kidney stones or ureteral stones.

In an embodiment, a shock wave and/or ultrasound device-which may be a lithotripter-includes an ultrasound and/or shockwave source (which, in one specific case, may be a single source judiciously configured to operate to produce ultrasound waves, shockwaves, or both). Such single source may be positioned within a reflector. Further, an ultrasound imaging sensor is provided, which may either be mounted at the ultrasound and/or shockwave source or may be configured as a separate unit, optionally made movable independently from the ultrasound and/or shockwave source.

The ultrasound imaging sensor is connected to a video processor and configured to deliver ultrasound imaging data to the video processor. The video processor is further connected to a display and/or a lithotripter control unit. The video processor includes a neural network (which is configured to detect and/or identify at least one kidney stone and/or at least one kidney). The neural network evaluates the X-ray or ultrasound image as an input signal, examines it for presence of images of known structures and then, if the probability of the kidney and/or stone is sufficient (that is, above a pre-determined threshold level), also marks them. The video processor may also be made a part of a so-called video matrix, which is configured to forward the above-mentioned input signals (videos or images) from the input device to at least one further monitor. Several monitors can also be connected to the output. This video matrix is appropriately structured to have enough processing power to examine the input images “on the fly” for the structures and then label and display them as an overlay if the probability is sufficient. This applies to the kidney as well as to a possible stone in the image.

The video processor is further configured to mark the at least one kidney stone and/or at least one kidney in at least one video image, which image may be then forwarded to the display and/or for providing position information of the at least one kidney stone and/or at least one kidney to the lithotripter control unit. In at least one case, the lithotripter control unit may be configured to position the ultrasound and/or shockwave source such as to place the at least one kidney stone substantially in the focus of the ultrasound and/or shockwave source. This may allow for automatic positioning of the shock wave or ultrasound device.

The embodiment helps a surgeon to identify a kidney, a ureteral stone, a kidney stone or other objects in an ultrasound image or video. The embodiment may also identify a bladder.

Detection of a stone may be improved by projecting a shadow of a stone into the image. This allows to increase the probability of detection.

The device may be configured to mark a stone or other concernment by superimposing closed curves on at least one image or a video.

For good ergonomics, the latency time of detecting and marking may be in a range of Is or shorter.

Herein, reference is made to the imaging data being video data or a video signal. Such a video signal contains a sequence of images. The disclosed device and method are also applicable for processing of a single image or a plurality of images.

Generally, beyond ultrasound video data, X-ray video data or X-ray images may be processed.

In a further embodiment, the video processor includes a neural network and a concernment detector. The neural network is configured for detection of at least one kidney in the video signal and forwarding of information of the at least one kidney to the concernment detector. The information about the kidney may include position information. The concernment detector may include an image processor and is configured to receive information about the kidney from the neural network. The concernment detector is configured to detect in the video signal at least one concernment or kidney stone at the position of the at least one kidney as indicated by the position information. Such a detection may be based on a shadow of a stone cast in an image.

The concernment detector may be configured to detect in the video signal at least one concernment or kidney stone by evaluating intensity distributions and an optional statistical evaluation. So, the probability of whether a certain object is a stone can be estimated and even indicated to a user.

The neural network may have been or is trained by input images showing different kidneys with and without concernments (stones). Together with a hexapod drive, the embodiments provide a significant improvement of shock wave and/or ultrasound therapy systems known from prior art. The hexapod drive provides superior flexibility in movement or positioning of the shock wave and/or ultrasound therapy system and/or an ultrasonic transducer for ultrasonic imaging. This allows to position the therapy system and/or the imaging system from different positions and different directions to a concernment or stone to be treated. So, a body part, e.g., ribs, the presence of which may degrade the ultrasonic view or the treatment, may be avoided. This embodiment works even better if the hexapod drive is coupled for appropriate automatic positioning.

In an embodiment, a shock wave and/or ultrasound device (which in at least one case may be a lithotripter) includes an ultrasound and/or shockwave source optionally positioned within a reflector. Further, an ultrasonic transducer for ultrasonic imaging may be coupled rigidly to the ultrasound and/or shockwave source. Such transducer may also be mounted within the reflector. The ultrasonic transducer may be arranged at the center of the ultrasound and/or shockwave source and may further be coaxial to the ultrasound and/or shockwave source. Alternatively, the ultrasonic transducer may be arranged at the center of the reflector and coaxially to the reflector. The ultrasonic transducer may further be oriented towards a focal volume of the shock wave and/or ultrasound device. Depending on the specifics of particular implementation, the ultrasonic transducer may be aligned parallel with the shock wave and/or ultrasound device. The ultrasonic transducer may be configured to generate an image of a part or of a significant portion or all of the focal volume of the shock wave and/or ultrasound device. A focal volume of the shock wave and/or ultrasound device may be indicated in the image of the shock wave and/or ultrasound device. This simplifies targeting or may help to verify automatic targeting. The ultrasonic transducer may be fixedly mounted within the reflector, such that it does not move relative to the reflector.

The ultrasound and/or shockwave source may be suspended on a hexapod drive, such as to be displaced in at least two or three degrees of freedom and tilted or rotated with one, two or three degrees of freedom. As the hexapod drive allows a free orientation of the source in space, such drive can basically adjust the ultrasound and/or shockwave source to any required position and orientation. The ultrasonic transducer moves together with the ultrasound and/or shockwave source. Therefore, the ultrasonic transducer can easily be moved for scanning a larger area by repositioning the ultrasound and/or shockwave source by means of the hexapod drive.

A controller may be provided to control the hexapod drive for specific movements of the ultrasonic transducer, e.g., for scanning a larger area. A larger area scan may include circular movements of the ultrasonic transducer. Further, the controller may be configured for automatically identifying and/or tracking of a concernment.

In an embodiment, 3D control device may be provided that allows a manual input for movements in 3D space. Such movements may be translation along two or three axes and tilt about one, two or three axes. In an embodiment, there may be implemented a translation in an X′-Y′ plane and rotation about a Z′-Axis, perpendicular to the X′-Y′ plane in a coordinate system related to the reflector, where the base of the reflector defines the X′-Y′ plane and the center axis of the reflector is along the Z′-Axis. The base of the hexapod may be in the X-Y plane of the system with its perpendicular Z-Axis of the shock wave and/or ultrasound therapy system. The data representing movements input by the 3D control device may be forwarded to the hexapod drive such that the ultrasonic transducer performs these movements. This allows for an easy movement of the ultrasonic transducer by a surgeon, for example for scanning a patient and/or for locating a concernment. The ultrasonic transducer may be moved by manual inputs to the 3D control device until the concernment is centered. Then the concernment will automatically be within the focal volume of the ultrasound and/or shockwave source. The controller may be configured to limit the movements of the hexapod drive in spatial relations and/or speed such that too wide or too fast movements are prevented. This may help to protect the patient.

The hexapod drive bearing the ultrasound and/or shockwave source is also known as a hexapod platform. Such a hexapod platform also is called a Stewart platform. Basically, it is a type of a parallel manipulator or parallel robot that has six linear actuators, which may be hydraulic or pneumatic jacks or electric linear actuators. Examples of electric linear actors are provided by motors coupled to a belt or a spindle to perform a linear movement. These linear actuators are connected in pairs to three mounting positions at a base, crossing over to three mounting positions at the ultrasound and/or shockwave source. Each connection of a linear actuator to either the base or the ultrasound and/or shockwave source may include a universal joint, also called a cardan joint or a ball joint. By varying the length of the linear actuators, the ultrasound and/or shockwave source can be moved in six degrees of freedom with respect to the base. There are three degrees of translation and three degrees of rotation. Although the use of six linear actuators is preferred, a lower number of actuators may be used such as, e.g., in a situation with a delta robot having three actuators.

The hexapod drive allows to adjust the position of an ultrasound and/or shockwave source together with the ultrasonic transducer relative to a patient's body and/or the X-ray system. Positioning of the ultrasound and/or shockwave source may be done automatically or by manual control. An automatic control may allow quick adjustment and it may also allow to store and retrieve preconfigured settings.

Such a hexapod drive is a very robust and mechanical stiff support or suspension of the ultrasound and/or shockwave source. Therefore, it can withstand high forces from the patient body, the weight of the ultrasound and/or shockwave source and dynamic loads which occur when shockwave pulses are generated. Further, a hexapod drive can be moved quickly. Therefore, a stable positioning of the ultrasound and/or shockwave source is achieved providing the additional ability to quickly correct deviations and movements by the patient.

A hexapod drive may allow movement in all 6 degrees of freedom. This allows for a precise adjustment of the position of the focal volume of the ultrasound and/or shockwave source and/or the path of the ultrasound and/or shockwave though the body of the patient.

There may be constraints in the movements, e.g., for safety reasons. In one example, the movement in Z direction may be limited to avoid injuring of the patient.

The ultrasound and/or shockwave source is oriented in a cartesian coordinate system as follows: a y-axis may be a longitudinal axis through the center of a patient table. An x-axis may be orthogonal to the y-axis and in the plane of the table surface. A z-axis is orthogonal to the plane of the table surface and therefore orthogonal to the x-axis and the y-axis and in a direction upward from the table. There may also be a first rotation around the x-axis, a second rotation and a third rotation around the z-axis. A positive rotation may be a clockwise rotation in a direction of a positive axis.

The shock wave or ultrasound therapy system may include a system controller which may control at least the hexapod drive of the ultrasound and/or shockwave source.

The ultrasound and/or shockwave source may be of any type of a source configured to generate shock waves. It may include a shock wave generator and/or transducer (which, in turn, may include at least one of a coil, a spark gap, or a Piezo transducer). The shock wave generator/transducer may be partially enclosed by a reflector. Depending on the type of transducer, the reflector may have a parabolic shape or substantially a shape of half of an ellipsoid. The ultrasound and/or shockwave source may have a focal volume which is distant from the ultrasound and/or shockwave source and normally around a center axis of the ultrasound and/or shockwave source. The focal volume may be defined as a volume, where the maximum shock wave intensity is maintained with a deviation of maximal −3 dB or −6 dB. If the focal volume is defined with a 6 dB deviation, the pressure at the limit of the zone is half of the maximum pressure inside the zone. The focal volume may have an ellipsoidal shape with a length in an axial direction (defined by the center axis) of the ultrasound and/or shockwave source axis of 10 to 15 cm and a diameter between 5 and 15 mm. The focal volume normally is spaced from the shock wave generator and/or transducer.

The patient table may have a substantially planar surface defining a longitudinal axis and configured for accommodating a patient thereon. The ultrasound and/or shockwave source may be mounted below the patient table. In general, a shockwave source may be mounted in alternative ways, e.g., on a stand or support.

### BRIEF DESCRIPTION OF THE DRAWINGS

In the following, the invention will be described by way of example, without limitation of the general inventive concept, on examples of embodiment and with reference to the drawings.

FIG. 1 shows an embodiment of a lithotripsy system.

FIG. 2 shows a side view of the embodiment of FIG. 1

FIG. 3 shows an embodiment schematically.

Generally, the drawings are not to scale. Like elements and components are referred to by like labels and numerals. For the simplicity of illustrations, not all elements and components depicted and labeled in one drawing are necessarily labels in another drawing even if these elements and components appear in such other drawing.

While various modifications and alternative forms, of implementation of the idea of the invention are within the scope of the invention, specific embodiments thereof are shown by way of example in the drawings and are described below in detail. It should be understood, however, that the drawings and related detailed description are not intended to limit the implementation of the idea of the invention to the particular form disclosed in this application, but on the contrary, the intention is to cover all modifications, equivalents and alternatives falling within the spirit and scope of the present invention as defined by the appended claims.

### DETAILED DESCRIPTION

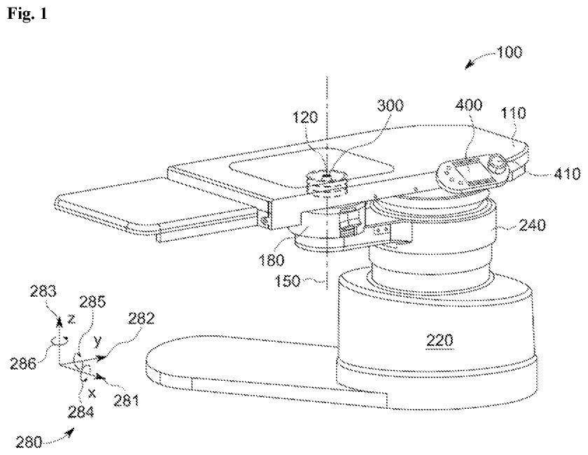

In FIG. 1, a first embodiment of a lithotripsy system is shown. (Generally, embodiments discussed herein are configured to work substantially with every ultrasound and/or shockwave treatment system).

An extracorporeal ultrasound and/or shockwave lithotripsy system for non-invasive treatment of stones 100 includes a patient table 110, an ultrasound and/or shockwave source 120 that includes an ultrasonic transducer 300 for ultrasonic imaging. The ultrasound and/or shockwave source 120 may be mounted to a hexapod drive 180. Herein, a hexapod mount and a hexapod drive is given as an exemplary embodiment. Actually, any other mount, e.g., a tiltable and rotatable mount may be used, as the embodiments are independent of the specific type of mount and would work with all known types of mounts. The hexapod drive 180 may further be held by a stand 220. The hexapod drive 180 allows fine positioning of the ultrasound and/or shockwave source 120 in multiple axes relative to the patient table 110 and therefore relative to the patient (not shown in this figure). The ultrasound and/or shockwave source 120 has a focal volume which may move together with the source. In an embodiment the distance of the focal volume to the source may be modified.

The patient table 110 may be based on a stand 220, which may stand on a floor. This stand may be the same or a different than the stand holding the shockwave source. The table 110 may be held by a positioning device 240 to move the table relative to the ultrasound and/or shockwave source 120 together with the hexapod drive 180. A movement of the patient table may be coarse positioning which may be limited to a displacement in 3 axes.

To describe relative movements of the table 110 and the ultrasound and/or shockwave source 120, a cartesian coordinate system 280 may be used. There is a y-axis 282, which may be a longitudinal axis 112 passing through the center of the table. Furthermore, there is an x-axis 281 orthogonal to the y-axis and in the plane of the table surface. A z-axis 283 is orthogonal to the plane of the table surface and therefore orthogonal to the x-axis and the y-axis. There may also be a first rotation 284 around the x-axis, a second rotation 285 and a third rotation 286 around the z-axis. A positive rotation may be a clockwise rotation in a view along a positive axis.

A hexapod drive 180 maybe structured to allow movement in each of and all of these 6 degrees of freedom. This allows for a precise adjustment of the position of the focal volume of the ultrasound and/or shockwave source 120. The patient table may be positioned for a slow and coarse adjustment in 3 degrees of translation for larger distances, but normally no rotation, whereas the hexapod drive provides a comparatively quick adjustment in 3 degrees of translation and 3 degrees of rotation. The movement distances of the table may be larger than the movement distances of the hexapod drive. An ultrasound device may at least be tiltable to perform a second rotation 285 around an axis parallel to the y-axis 282. It may also be tiltable around an axis parallel to the Y axis. It may further be translated in a plane defined by the X and Y axis. This means, it may be moved on the floor which is also in the X-Y plane. Further, it may be rotated along the Z-axis of the shockwave source 120.

For control of the movement of the ultrasound and/or shockwave source 120 relative to the patient table 110, a control panel 400 may be provided, which may include a 3D control device 410, e.g., a control button.

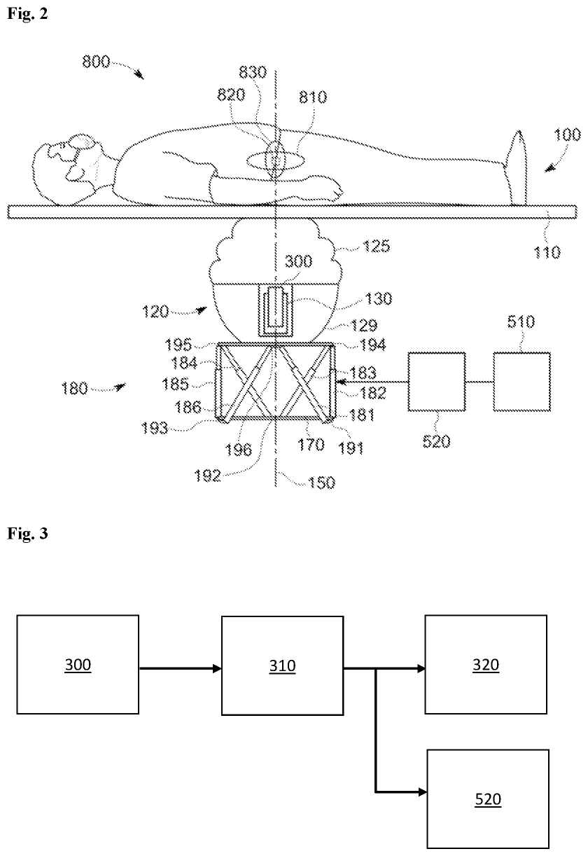

FIG. 2 shows a side view. A shock wave or ultrasound device, which may be a lithotripter 100 may include a patient table 110 having a flat surface and an ultrasound and/or shockwave source 120. The ultrasound and/or shockwave source 120 which is shown in a sectional view may be arranged below the patient table 110, such that a patient 800 may be accommodated on top of the patient table. The patient may have a kidney 810 with a kidney stone 820.

A shock wave generator 130 may be held within a reflector 129. In this embodiment, the shock wave generator 130 may include a coil. Centered to the shock wave generator 130 an ultrasonic transducer for ultrasonic imaging 300 may be provided. It may be aligned with the center axis of the ultrasound and/or shockwave source. Such an ultrasonic transducer for ultrasonic imaging may basically be mounted together with the ultrasound and/or shockwave source 120 at the hexapod drive 180, such that both components may be moved together. The ultrasonic transducer for ultrasonic imaging may be configured to generate an image of a part or all of a focal volume 830 of the shock wave and/or ultrasound source 120.

A system controller 510 may be provided to control all system functions. It may also receive input from the control panel 400 and provide user feedback via the control panel. A hexapod control 520 may receive input from the system controller 510 and control the individual actuators of the hexapod drive.

FIG. 3 shows a schematic diagram. An ultrasonic transducer for ultrasonic imaging 300 or an X-ray imaging system provides a video signal (imaging data) to a video processor 310. The video processor includes a neural network which is configured to detect and/or identify and to mark in the video signal at least one kidney stone and/or at least one kidney itself. The video signal with markings is delivered to a display 320. Further position and/or orientation data of the at least one kidney stone and/or at least one kidney may be delivered to a system controller 510 for positioning the ultrasound and/or shockwave source 120. This may allow for automatic positioning of the shock wave or ultrasound device.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. In one specific case, the terms “approximately”, “substantially”, and “about”, when used in reference to a numerical value, represent a range of plus or minus 20% with respect to the specified value, more preferably plus or minus 10%, even more preferably plus or minus 5%, most preferably plus or minus 2% with respect to the specified value.

The term “A and/or B” or a similar term is defined to be interchangeable with the term “at least one of A and B.” The term “image” refers to and is defined as an ordered representation of detector signals corresponding to spatial positions. For example, an image may be an array of values within an electronic memory, or, alternatively, a visual or visually-perceivable image may be formed on a display device such as a video screen or printer.

It will be appreciated to those skilled in the art having the benefit of this disclosure that this invention is believed to provide a Lithotripter with Detection of Urethral Stones.

Further modifications and alternative embodiments of various aspects of the invention will be apparent to those skilled in the art in view of this description. Accordingly, this description is to be construed as illustrative only and is provided for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

### LIST OF REFERENCE NUMERALS

100 shock wave and/or ultrasound therapy system
110 patient table
120 ultrasound and/or shockwave source
129 reflector
130 shock wave generator
150 center axis
170 base
180 hexapod drive
181-186 linear actuators
191-193 mounting positions at base
194-196 mounting positions at ultrasound and/or shockwave source
220 stand
240 positioning device
280 cartesian coordinate system
281 x-axis
282 y-axis
283 z-axis
284 tilt around the x-axis
285 tilt around the y-axis
286 tilt around the z-axis
300 ultrasonic transducer for ultrasonic imaging
310 video processor
320 display
400 control panel
410 3D control device
510 system controller
520 hexapod control
800 patient
810 kidney
820 kidney stone
830 focal volume

1. A shock wave and/or ultrasound therapy system comprising:
an ultrasound and/or shockwave source that includes an ultrasonic transducer for ultrasonic imaging or an X-ray imaging system, wherein
the ultrasonic transducer or an X-ray imaging system that is configured to provide a video signal and that is coupled to a video processor, the video processor including a neural network and a concernment detector,
wherein the neural network is configured to detect at least one kidney in the video signal and forwarding of position information of the at least one kidney to the concernment detector, and
wherein the concernment detector is configured to detect in the video signal at least one concernment or kidney stone at a position of the at least one kidney.

2. The shock wave and/or ultrasound therapy system according to claim 1, wherein the video processor is configured to mark in the video signal the at least one kidney stone and/or at least one kidney itself, and wherein the video processor video signal is coupled to a display to deliver the video signal with markings to the display.

3. The shock wave and/or ultrasound therapy system according to claim 1, wherein the video processor is configured to deliver position data and/or orientation data of the at least one kidney stone and/or at least one kidney to a system controller configured to position the ultrasound and/or shockwave source.

4. The shock wave and/or ultrasound therapy system according to claim 1, wherein the video processor includes:
a first section containing the neural network, the neural network being configured to detect the at least one kidney, and
a second section containing an image processor that is configured to detect a concernment or kidney stone based on information about the at least one kidney received from the first section.

5. The shock wave and/or ultrasound therapy system according to claim 1, wherein
the neural network has been trained by input images showing different kidneys with and without concernments and/or stones.

6. The shock wave and/or ultrasound therapy system according to claim 1, wherein
the concernment detector is configured to detect in the video signal at least one concernment or kidney stone by evaluating intensity distributions in said video signal.

7. The shock wave and/or ultrasound therapy according to claim 1, wherein the ultrasound and/or shockwave source is suspended on a hexapod drive.

8. The shock wave and/or ultrasound therapy system according to claim 1, wherein the ultrasonic transducer for ultrasonic imaging or the X-ray imaging system is coupled rigidly to the ultrasound and/or shockwave source and/or the hexapod drive such that movements of the hexapod drive are directly coupled to the ultrasonic transducer.

9. The shock wave and/or ultrasound therapy system according to claim 1, wherein the ultrasonic transducer for ultrasonic imaging or the X-ray imaging system is arranged:
at the center of the ultrasound and/or shockwave source and coaxially to the ultrasound and/or shockwave source,
or
at the center of the reflector and coaxially to the reflector.

10. The shock wave and/or ultrasound therapy system according to claim 1, wherein
(10A) the ultrasonic transducer for ultrasonic imaging or the X-ray imaging system is aligned parallel with the ultrasound and/or shockwave source, and/or
(10B) the ultrasonic transducer for ultrasonic imaging or the X-ray imaging system is oriented towards a focal volume defined by the shock wave and/or ultrasound device.

11. The shock wave and/or ultrasound therapy system according to claim 1, wherein the ultrasonic transducer for ultrasonic imaging or the X-ray imaging system is configured to generate an image of a part or all of a focal volume of the shock wave and/or ultrasound source.

12. The shock wave and/or ultrasound therapy system according to claim 1, further comprising a hexapod drive, wherein the hexapod drive includes six linear actuators that are attached in pairs to three positions at a base of the hexapod drive, crossing over to three mounting positions at the ultrasound and/or shockwave source.

13. The shock wave and/or ultrasound therapy system according to claim 1, wherein each connection of linear actuators present in the system to either a base of a hexapod drive of the system or the ultrasound and/or shockwave source includes a universal joint or a ball joint.

14. The shock wave and/or ultrasound therapy system according to claim 1, wherein linear actuators present in the system include at least one of a hydraulic actuator or a pneumatic jack or an electric linear actuator.

15. The shock wave and/or ultrasound therapy system according to claim 1, further comprising a hexapod drive configured to provide movement with at least five degrees of freedom including displacement along three orthogonal axes and at least two degrees of tilt.

16. The shock wave and/or ultrasound therapy system according to claim 6, wherein the concernment detector is configured to detect in the video signal at least one concernment or kidney stone with the use of statistical evaluation of said video signal.

17. The shock wave and/or ultrasound therapy system according to claim 11, wherein the ultrasonic transducer for ultrasonic imaging or the X-ray imaging system is configured to generate said image that contains indicia of a focal volume of the shock wave and/or ultrasound device.
